(19) United States
(12) Patent Application Publication
(10) Pub. No.: US 2022/0022782 A1
(43) Pub. Date: Jan. 27, 2022

(54) Apparatus and Methods for Wirelessly Powered Biosensors

(71) Applicant: The Regents of the University of California, Oakland, CA (US)

(72) Inventor: Aydin Babakhani, Los Angeles, CA (US)

(73) Assignee: The Regents of the University of California, Oakland, CA (US)

(21) Appl. No.: 17/383,711

(22) Filed: Jul. 23, 2021

Related U.S. Application Data
(60) Provisional application No. 63/056,148, filed on Jul. 24, 2020.

(51) Int. Cl. A61B 5/1473; A61B 5/145; H02J 50/00; H02J 50/10; H02J 50/27; H02J 50/80

(52) U.S. Cl. CPC A61B 5/1473; A61B 5/14532; A61B 5/14546; A61B 2560/0219; H02J 50/10; H02J 50/27; H02J 50/80; H02J 50/001

(57) ABSTRACT

Wirelessly powered biosensors are described. In an embodiment, a wirelessly powered electrochemical sensor system includes: an external controller, an implantable microchip that includes an electrochemical sensor coupled to a target molecule, where the microchip communicates with the external controller and receives wireless power from the external controller, where the microchip measures at least one of current and voltage from the electrochemical sensor.

### CROSS REFERENCE TO RELATED APPLICATIONS

This application claims priority to U.S. Provisional Application Ser. No. 63/056,148, entitled “Apparatus and Methods for Wirelessly Powered Biosensors” to Babakhani, filed Jul. 24, 2020, the disclosure of which is herein incorporated by reference in its entirety.

### FIELD OF THE INVENTION

The present invention generally relates to wireless biosensors, and in particular to Point of Care (PoC) electrochemical immunosensors for detection of IgM and IgG antibodies related to COVID-19 and biosensors for glucose monitoring.

### BACKGROUND

Many different health issues, both new and ongoing, are affecting different cross sections of the world population. Currently, the COVID19 pandemic has surpassed 8.2 million reported cases, according to Johns Hopkins University, and 444,437 deaths with America being one of the worst hit countries that has reached over 2.2 million known infections and 119,000 deaths (Jun. 16, 2020). To date, the testing for the virus is still limited and/or slow in many areas in the United States.

Currently, the most effective way to diagnose whether COVID-19 is infecting an individual is using Real-Time Polymerase Chain Reaction (RT-PCR), which is recognized as a gold standard by the WHO. RT-PCR is an extremely sensitive nucleic acid-based test, usually done using a nasal swab, or more recently by using saliva. RT-PCR detects the genetic material of the virus to indicate whether an infected person is carrying the virus and might be contagious. On the other hand, a blood draw of a person can be analyzed to indicate whether (s)he has mounted an immune response to the disease-causing virus through a serological test indicating whether a person was infected earlier—but possibly, no longer contagious.

Both categories of SARS-CoV-2 tests, namely the nucleic acid tests and the serological tests can be employed retrospectively to determine the true scope of the pandemic and assist in the calculation of statistics, including the fatality rates. Point-of-Care (POC) devices for both types of tests exist but require significant operator involvement—and are therefore performed in facilities that are self-contained. These tests also need the use of special equipment to prevent operator infection or sample test contamination from air-borne transmission of contagious viruses. However, the order of specimen collection, shielding, and sample transferring to the laboratory are indispensable for this test and place all individuals nearby at risk of infection since the virus can occur through air-borne transmission. Also, a commercial PCR-based diagnosis is comparably expensive since it is necessary to require lab equipment and technical expertise.

Certain ongoing health issues also share similar deficiencies. For example, currently diabetes (caused due to deficiency of insulin and resulting in abnormal blood glucose (BG) levels) significantly affects nearly 500 million people. It leads to dire consequences, including kidney failure, strokes, heart attacks, high blood pressure, blindness, and coma. Diabetes was one of the leading causes of death in the United States. Diabetes mellitus is broadly classified into two groups, i.e., Type 1 and Type 2, where abnormal BG levels are caused by the absence of insulin secretion (Type1) and abnormal insulin secretion (Type 2) in the human body. As per CDC report 2020, epidemic diabetes affects 120 million people in the U.S., including an increasing population of children under 18. Among those affected populations, approximately 32.5 million have Type 2 diabetes, 1.71 million have Type 1 diabetes, and the remaining 88 million have prediabetes or impaired glucose tolerance (IGT).

### BRIEF SUMMARY OF THE INVENTION

Systems and methods for wirelessly powered biosensors are described. In an embodiment, a wirelessly powered electrochemical sensor system includes: an external controller, an implantable microchip that includes an electrochemical sensor coupled to a target molecule, where the microchip communicates with the external controller and receives wireless power from the external controller, where the microchip measures at least one of current and voltage from the electrochemical sensor.

In a further embodiment, the harvested wireless power is stored in a capacitor.

In a further embodiment, the stored energy in the capacitor is used to generate a regulated voltage to activate the electrochemical sensor.

In a further embodiment, the harvested wireless power is received by at least one of a loop antenna, dipole antenna, resonant loop antenna, inductive coupling, and resonant inductive coupling.

In a further embodiment, a frequency of the wireless power is between 1 MHz and 1 GHz.

In a further embodiment, the physical distance between the implantable microchip and external controller is smaller than a wavelength of the electromagnetic signal used for wirelessly powering the microchip.

In a further embodiment, a plurality of commands are sent by the external controller and are encoded on the RF carriers by means of amplitude shift keying (ASK), frequency shift keying (FSK), phase shift keying (PSK), on-off keying (OOK), or other complex coding schemes.

In a further embodiment, an electrochemical signature measured by the implantable microchip is transmitted wirelessly to the external controller.

In a further embodiment, the electrochemical signature is digitized by an ADC before being transmitted to the external controller.

In a further embodiment, the electrochemical sensor is a glucose sensor that monitors glucose levels.

In a further embodiment, the glucose sensor includes: an electrochemical glucose sensor with an implantable electrode with a three-electrode setup; a readout interface that responds electrically to redox reaction with glucose; an interface that processes, stores, and transmits signals wirelessly from subcutaneous tissue of the patient.

In a further embodiment, the electrochemical glucose sensor is a 4-electrode setup, where 3 electrodes in the setup test electrochemicals and a 4^{th} electrode in the setup is at least one of a glucose sensor and lactate sensor that generates simultaneous measurements.

In a further embodiment, the wirelessly powered electrochemical sensor system further includes: an on-chip analog to digital converter (ADC); a potentiostat amplifier that maintains a fixed voltage between working and reference electrodes in the three-electrode setup equal to the potential of the electrochemical cell; where the potentiostat amplifier measures current flowing through the electrochemical cell, converts the current to voltage, and boosts the voltage to drive the on-chip ADC.

In a further embodiment, the wirelessly powered electrochemical sensor system further includes an on-chip instrumentation amplifier that amplifies a low-frequency output voltage of implantable electrode, where an analog output of the on-chip instrumentation amplifier is converted to digital bits using the on-chip ADC.

In a further embodiment, the external controller is positioned on or near a patient's skin and the implantable microchip is implanted beneath the skin.

In a further embodiment, the external controller is an application executing on a mobile device.

In a further embodiment, the wirelessly powered electrochemical sensor system further includes applying a voltage between a reference electrode and a working electrode while reading a current from a counter electrode.

In a further embodiment, the wirelessly powered electrochemical sensor system further includes sweeping the voltage and reading of the current to plot a current versus voltage curve, where the curve is used to identify different biomolecules.

In a further embodiment, the microchip further includes: an on-chip RF-power receiving antenna that receives electromagnetic power from the external controller; an on-chip transmitting antenna that transmits data to the external controller; a power management unit (PMU) that includes a capacitor to rectify and store the electromagnetic power, where the PMU monitors harvested energy and operates the electrochemical sensor.

In a further embodiment, the electrochemical sensor detects COVID-19 reactive antibodies of the IgM and IgG isotypes.

### BRIEF DESCRIPTION OF THE DRAWINGS

FIG. 1 illustrates readout of results and Calobratio in accordance with an embodiment of the invention.

FIG. 2 illustrates a circuit schematic of an amplifier in accordance with an embodiment of the invention.

FIG. 3 illustrates a block diagram of a wirelessly powered transceiver in accordance with an embodiment of the invention.

FIG. 4A illustrates a circuit schematic of a UWB TX in accordance with an embodiment of the invention.

FIG. 4B illustrates a circuit schematic of an ASK data RX in accordance with an embodiment of the invention.

FIG. 5 illustrates a block diagram of a high-performance biochemical sensor in accordance with an embodiment of the invention.

FIG. 6A illustrates a fully wireless and batteryless continuous glucose monitoring (CGM) system in accordance with an embodiment of the invention.

FIG. 6B illustrates fabricating custom miniaturized fully functioned batteryless and wirelessly pacemakers with needle-type electrodes in rabbits and open chest porcine animal models in accordance with an embodiment of the invention.

FIG. 6C illustrates a wireless system with PCB and output data in accordance with an embodiment of the invention.

FIG. 7 illustrates a circuit schematic of potentiostat amplifier in accordance with an embodiment of the invention.

FIG. 8 illustrates a microchip architecture of an implantable CGM in accordance with an embodiment of the invention.

FIG. 9 illustrates a circuit architecture of an electrochemical sensor platform with a square-wave generator in accordance with an embodiment of the invention.

FIG. 10 illustrates a circuit architecture of a square wave generator in accordance with an embodiment of the invention.

FIG. 11 illustrates a circuit architecture of a potentiostat in accordance with an embodiment of the invention.

FIG. 12 illustrates a circuit architecture of an electrochemical sensor in accordance with an embodiment of the invention.

### DETAILED DESCRIPTION OF THE DRAWINGS

Turning now to the drawings, systems and methods for wireless electrochemical biosensors in accordance with embodiments of the invention are illustrated in several embodiments, the biosensor is a wireless, sensitive, and disposable Point of Care (PoC) electrochemical immunosensor for detection of glucose, IgM and/or IgG antibodies related to COVID-19 and/or other diseases including diabetes among others. Accordingly, many embodiments of the invention provide a wireless, disposable, and sensitive electrochemical immunosensor that can indicate the presence of COVID-19 infection, or an individual's immune response to the infection in detail from samples of test patients. Described below are details regarding designs of electrochemical immunosensor electrode interfaces to detect and/or monitor various diseases, including, among others, diabetes and/or COVID-19 reactive antibodies of the IgM and IgG isotypes. Furthermore, described below are design details regarding the development of wireless and battery-free readout electronics to enable sensitive and low-cost IgG/IgM detection. Many embodiments enable a low-cost small form-factor. In many embodiments, an ultra-low power data transceiver can be fabricated and validated for an entirely wireless interface.

In many embodiments, the Minimum Viable Product (MVP) may be a wireless electrochemical immunosensor platform with high expandability and applicable to many transducers that rely on current/voltage measurements. In many embodiments, the biosensor can be integrated into a miniaturized, fully wireless platform, resulting in rapid, reliable, and massively scalable fabrication, to create an inexpensive detection system that may not require an operator to be potentially exposed to the contagious virus through its wireless functionality—making it easily amenable to high throughput and robotic control.

### COVID 19 Overview

COVID-19 shows strong contagion with multiple respiratory diseases, including fever, cough dyspnea, and viral pneumonia. To prevent the rapid spread of the COVID-19 virus, the movement of a large number of citizens has been limited, and social distancing has slowed down the viral spread, but this is an insufficient method which does not provide a long-term solution to the problem.

Serological tests detect antibodies present in the blood when the body is responding to a specific infection, like COVID-19. They detect the body's immune response to the infection caused by the virus rather than detecting the virus itself. In the early days of an infection, when the body's immune response is still building, antibodies may not be present in detectable levels. This limits the test's effectiveness for diagnosing COVID-19. Currently authorized serological tests for SARS-CoV-2 measure IgM and/or IgG antibodies but they lack enough sensitivity. Most of these tests do not provide any quantitative level of antibodies and only show a color change on a strip when a high level of a specific antibody exists.

During the Exposure Phase (recent infection)—there is a high viral load that can only be detected by a nucleic acid test (typically 2 to 3 weeks). Subsequently, the Acute Phase is initiated, which consists of the viral load being cleared and no longer detectable by the nucleic acid test (about 3 weeks post-infection), but it is possible to start to see the immune response in the form of antibody production. Typically, it can be an IgM response about 5 days after symptoms begin to appear. Finally, during the Antiviral Response, there is an increase in the production of both IgM and IgG antibodies that can reach to a high level (˜100%) in 15 days after symptoms appear. These antibody levels can stay high for 6 months or more after the infection.

### Biosensor Designs

Through biospecimen sampling and application, many embodiments of the biosensor platform provide a platform that allows patients with a fully automated detection mechanism and without the need or involvement of a trained technician, thus reducing the risk of transmission. Many embodiments of the biosensor platform thus provide a means for people to know if they are infected (or were infected) with COVID-19 due to the high level of IgG/IgM in their samples. The biosensor platform in accordance with many embodiments can be easily accessed by many people rapidly and inexpensively. Many embodiments of the biosensor platform provide for wireless electrochemical sensor readout circuits with high adaptability, low-power, and low-noise for robust and quantitative sensing of COVID-19 antibodies. In many embodiments, the biosensor platform can be designed as a fully battery-less system with on-site sensing capability, so that risk of sample exposure can be reduced. In many embodiments, the biosensor platform can be based on silicon technology allowing for mass production at low cost.

### Biosensor Design Architecture

Many embodiments of the biosensor platform leverage silicon-based integrated sensors, communication circuits, and wireless power transfer technologies to develop a platform that can be translated into Point-of-Care (POC) devices being used for COVID-19 diagnosis and surveillance with human bio-specimen samples. In particular, biosensors can be classified according to the transducer working principle, into optical, electrochemical, piezoelectric, or magnetic. Many embodiments of the biosensor platform provide a biosensor that is an (a) electrochemical immunosensor coupled to the target molecule, (b) a readout interface that responds electrically to target binding, and (c) an interface that processes, stores, and transmits the signals wirelessly.

### Designs of Electrochemical Immunosensor Electrode Interface

Many embodiments of the biosensor provide a design for an electrode interface with an Ag(I)-cysteamine complex based electrochemical stripping immunoassay, that provides ultrasensitive human IgG detection using an Ag (I)-cysteamine complex (Ag-Cys) as a label. In many embodiments, the sensing can consume as low an amount of sample as possible and can be scalable, rapid, and inexpensive for the detection of multiple biomarkers. An Ag-Cys complex-based immunosensor can be fabricated for the ultrasensitive detection of IgG. The immunosensor can be fabricated by immobilizing an anti-IgG antibody on to a conducting polymer layer on a glassy carbon electrode. The target protein, IgG is sandwiched between anti-IgG antibody and AuNPs conjugated anti-IgG anti-body. The Ag-Cys complex can then attached to the AuNPs-anti-IgG through the adsorption of amine groups of cysteamine on the AuNPs. The surface of the Ag-Cys complex-based immunosensor was characterized using QCM and XPS. The Ag-Cys complex-based immunosensor exhibited a wide dynamic range and a low detection limit of (0.4 fg/mL). The Relative Standard Deviation (RSD) value was determined to be 3.8% at the IgG concentration of 10⁻¹³ g/mL (0.1 pg/mL). The interference-free and the excellent stability of the present Ag-Cys complex-based immunosensor can be a useful method for clinical purposes. Accordingly, many embodiments fabricate an electrochemical immunosensor by immobilizing anti-IgG antibody on a poly-5,2′: 5′,2″-terthiophene-3′-carboxylic acid (polyTTCA) film grown on the glassy carbon electrode through the covalent bond formation between amine groups of anti-IgG and carboxylic acid groups of polyTTCA.

Then the target protein, which can be the IgG molecule itself, can be sandwiched between an anti-IgG antibody that had been covalently attached onto the polyTTCA layer and the labeled detector anti-IgG antibody that can be created as a gold nanoparticles (AuNPs) conjugated human immunoglobulin G (anti-IgG) antibody (AuNPs-anti-IgG).

In many embodiments, the bound AuNP-ant-iIgG can be tagged using an Ag (I)-cysteamine complex (Ag-Cys) as a label.

In many embodiments, by using square wave voltammetry, each target concentration can be quantified through the Ag stripping voltammograms. Many embodiments target a wide dynamic range with the detection limit of 0.4±0.05 fg/mLT to human IgG spiked serum samples.

Many embodiments provide an immunoassay based on the above protocol for the determination of both IgG and IgM levels against COVID-19 viral antigens present in human serum samples by fabricating a (COVID-19) antigen capture sandwiching site.

Described below are details on electrode fabrication in accordance with many embodiments of the biosensor platform.

Step 1—Antigen Capture Using COVID-19 Sandwiching Site: Preparation of anti-IgG and anti-IgM capture platforms using an Ag-Cys complex-based immunosensor can be created by immobilizing a selected, commercially available anti-COVID-19 from polyclonal antisera to the COVID-19 Virus (Anti-COV-19 PK) onto a conducting polymer layer on glassy carbon electrode (that may be used for both IgM and IgG detection subsequently).

Step 2—Immobilizing functional COVID-19 as an Anchoring Virions: Viral COVID-19 (Anchoring virus), inactivated to be non-contagious, can be the immobilized onto the Antigen Capture Sandwiching Site (made in Step 1).

Step 3—Addition of Biospecimen Sample being Tested: The target protein(s) that need to be measured, which in this example may be the antibodies that are specifically reacting against COVID-19 virion surface protein from clinical serum samples can be allowed to interact with immobilized COVID-19 virions.

Step 4—Binding of anti IgM and IgG probes previously coupled with AuNP: AuNP conjugated to anti-human IgG antibody (AuNP-anti-IgG) and, separately, in parallel, and in another chamber, another probe for detecting IgM isotype (AuNP-anti-IgM) can be added and incubated to allow specific binding between each isotype and its specific detection antibody coupled to AuNP.

Step 5—Addition of Ag (I)-cysteamine complex (Ag-Cys) as a label: The tag consisting of Ag-Cys complex can then be allowed to attach to the bound AuNPs-anti-IgG (or AuNps-anti-IgM) through the adsorption of amine groups of cysteamine on the AuNPs.

Alternatives: The detection limits and sensitivity and specificity profiles inherent in these assays can depend on the polyclonal antisera used for immobilizing the COVID-19 antigen. For example, the polyclonal sera could bind to the COVID-19 antigen at a site that is antigenic and therefore would be unavailable to the reactive human IgM or IgG. This problem can be addressed by using an appropriate monoclonal to capture the COVID-19 virion. Serum samples can contain interfering substances in sensitive ELISA assays and often, a precalculated dilution of the starting sample that gives an optimal signal/noise ratio needs to be evaluated prior to the actual serum measurements from biospecimens.

Analytical calibration, specificity, and sensitivity of the assays can be performed by using non cross-reactive coronavirus strains in the antigen capture steps. The diagnostic capabilities of the system and the ability to determine the lower limit of viral protein numbers corresponding to the assay (compared to the anticipated 0.4 fg/mL range of published values for IgG target antigen spiked into human serum) can be determined as the assays get validated.

### Design of Wireless and Battery-Free Readout Electronics for Sensitive IgG/IgM Detection.

Integration of a biochemical sensor to a portable and miniaturized biosensor platform in accordance with many embodiments can reduce human errors and allow convenient use of sensing platform as a point-of-care application. Many embodiments of the biosensing platform can provide an inexpensive and disposable miniaturized kit to thereby accelerate mainstream adoption of the sensing platform. Many embodiments of the biosensing platform can provide for wireless operation which can eliminate the use of any wire between a disposable kit and a reader/analyzer.

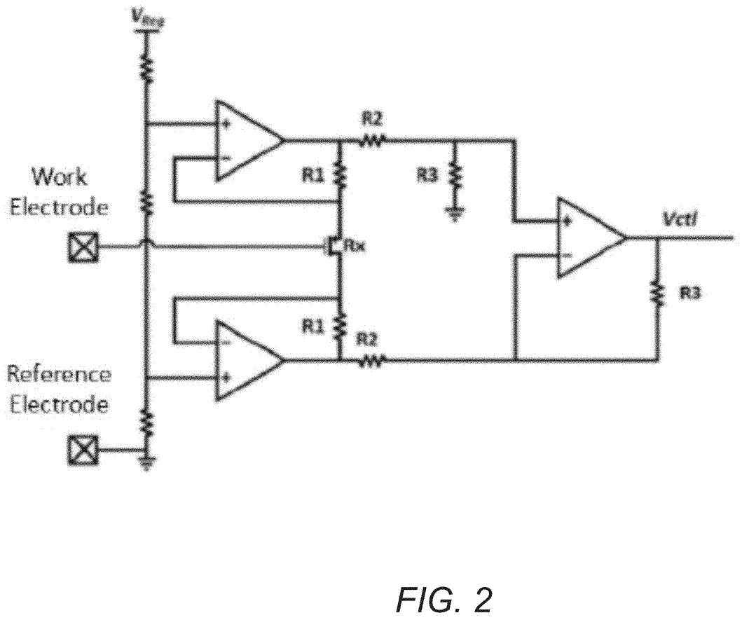

Many embodiments of the biosensing platform provide an integrated signal conditioning and data communication microchip that can include the following building blocks: (1) an integrated instrumentation amplifier; (2) a high-resolution Analog to Digital Converter (ADC) (3) a low-power Parallel Input Serial Output (PISO) memory; (4) an ultra-low power data receiver and a clock recovery sub-system; and (5) an ultra-low power data transmitter. In many embodiments, the microchip can be integrated and attached to the electrode interface. The electrode/microchip assembly in accordance with many embodiments can be low cost (<$1) and disposable. In many embodiments, the biochemical electrodes that are utilized for virus detection can have low driving current and high output impedance. Hence, the output signal of the electrode may not be directly used for driving data communication circuitry that loads the preceding stages. Also, the ambient noise sources of the power link can overwhelm the output signal of the electrode and thus invalidate the experiment. As a remedy to this problem, many embodiments provide a low-power amplifier to boost the output signal of the electrode and convert it to an acceptable level for driving an ADC. Many embodiments provide for a fully differential design, whereby the amplifier can achieve a Common Mode Rejection Ratio (CMRR) of −60 dB, which may result in the reduction, if not complete elimination, of the common-mode noise sources. To amplify the low-frequency signal of the electrode, many embodiments implement an on-chip instrumentation amplifier with a block diagram that is shown in FIG. 2 in accordance with an embodiment of the invention. The design in accordance with many embodiments of the invention can be used to implement an integrated pH sensor achieving a variable gain of up to ×50 with a power consumption of less than 10 μW. Although FIG. 2 illustrates a particular on-chip amplifier architecture, any of a variety of architectures may be specified as appropriate to the requirements of specific applications in accordance with embodiments of the invention.

Many embodiments of the biosensor platform can enable low power wireless communication, where the analog output of the on-chip instrumentation amplifier can be converted to digital bits to preserve the accuracy of data during transmission. Many embodiments may include a low-power on-chip ADC with 10-bit resolution to enhance the overall detection accuracy of the biochemical sensor. By adopting a set-and-down architecture, many embodiments can shrink the overall size of the ADC in half. The ADC in accordance with many embodiments can be implemented with a unit capacitor size of 17.5 fF achieving a power consumption of 300 nW for a sampling rate of 10 KSps. Simulated results show that the designed ADC in accordance with many embodiments achieves a Signal-to-Noise-Distortion-Ratio (SNDR) of 67.8 dB and an Effective Number of Bits (ENoB) of 9.3. In many embodiments, the digitized data can be loaded in a PSIO memory and fed to the data transmitter serially. The PSIO can be implemented as a digital shift register.

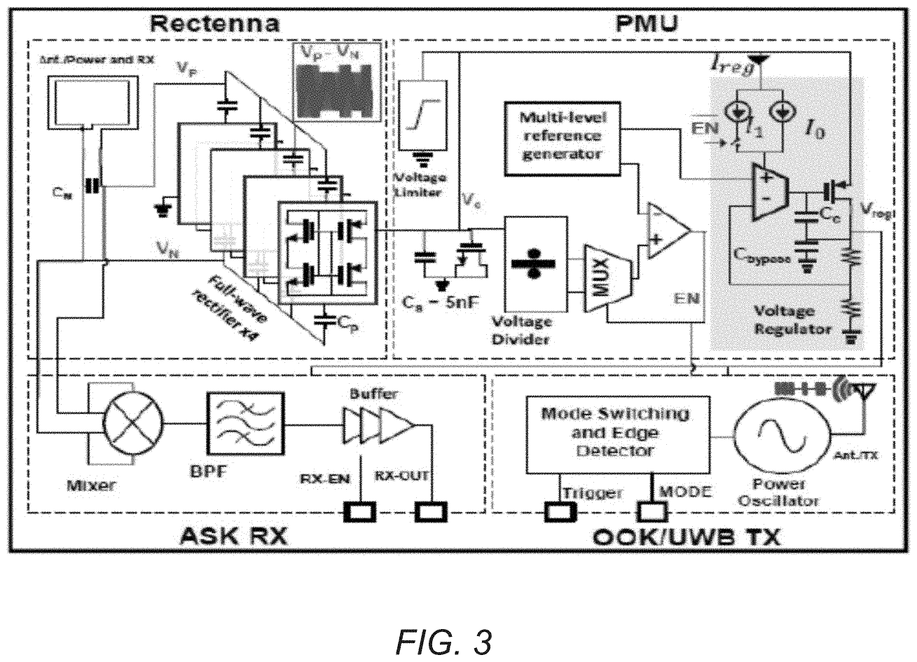

On-chip wireless radio can be a critical task that dominates the power consumption and limits the operating range. Active radios have been demonstrated with >10 Mbps data rate and >5 cm operating range, recently. However, off-chip components that are used for power delivery and data communication have limited integration capability of these devices and there can be a need for developing an integrated radio with a mm sized form factor integrated on the chip. Accordingly, many embodiments provide a fully integrated wireless sensor. In particular, many embodiments provide for an integrated wirelessly powered radio equipped with on-chip antennas that may consume a total area of 2.4×2.2×0.3 mm³ achieving 2.5 Mbps downlink (DL) DR with 1.04 pJ/s efficiency, maximum uplink (UL) DR of 150 Mbps under duty-cycled operation, and a maximum UL energy efficiency of 4.65 pJ/b. An architecture of a wirelessly powered transceiver in accordance with an embodiment of the invention is shown in FIG. 3. The robust operation in accordance with many embodiments of the invention can enabled by the techniques described below.

Many embodiments can co-optimizing the on-chip coil (OCC) and wireless link with voltage rectifier to maximize the power transfer efficiency. Many embodiments can combine MOSCAP (5.5 fF/μm2) and MIM (2 fF/μm2) capacitors to realize a ˜5 nF on-chip capacitor for energy storage. Many embodiments can employ a power management unit (PMU) to set the operating mode and biasing condition of different blocks depending on the available power and power consumption of the system. Certain embodiments may utilize a dual-antenna architecture to minimize the interference between power link and UL communication. Many embodiments can exploit amplitude-based modulation schemes in UL and DL for maximizing energy efficiency and utilizing an architecture based on a power oscillator (PO) in the transmitter (TX) block to achieve the highest possible energy efficiency. Although FIG. 3 illustrates a particular circuit architecture of a wirelessly powered transceiver, any of a variety of circuit designs may be specified as appropriate to the requirements of specific applications in accordance with embodiments of the invention.

Many embodiments of the biosensor platform can equip a sensor with a clock recovery circuitry to provide a reliable clock signal for the ADC and digital blocks. In addition, many embodiments are able to increase the communication distance to >2 m by improving the efficiency of the UL path. Electromagnetic simulation results reveal that the UL efficiency for a 2 m distance between a chip and an external receiver is about −81 dB. The minimum transmitted power level from the chip can be estimated from (1) assuming a receive bandwidth of 10 KHz, a Noise Figure (NF) of 3 dB and a minimum SNR of 10 dB.

P_{T,min}=−174 dBm/Hz−ηₗᵢₙₖ+SNR+10×log(BW)+NFᵣ=−174+81+10+40+3=−40 dBm (Equation 1)

According to (Equation 1), the required transmitted power level from the chip for achieving a 2 m operating distance should be about 0.1 μW which may be much lower than the available power budget (10 Hence, the data communication can also be conducted continuously and there may be no need for the duty-cycling.

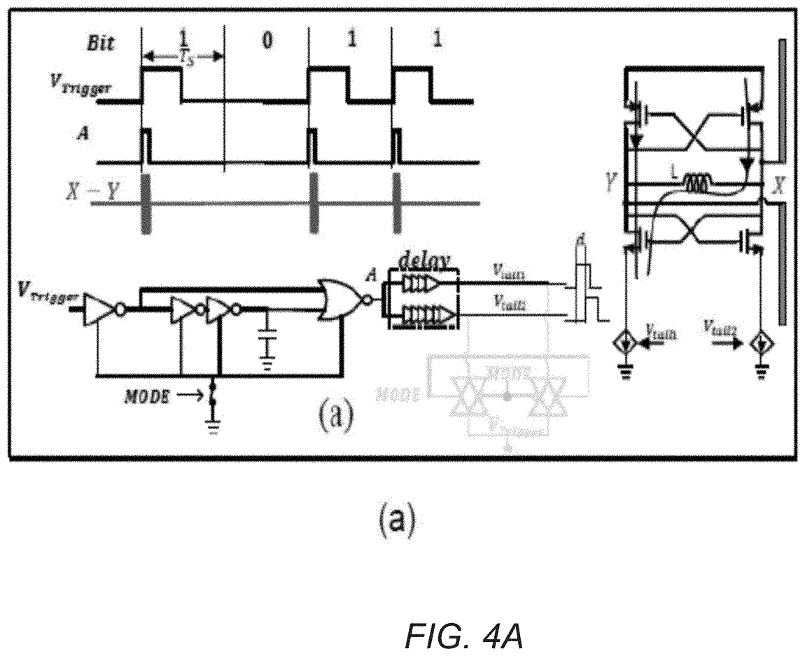

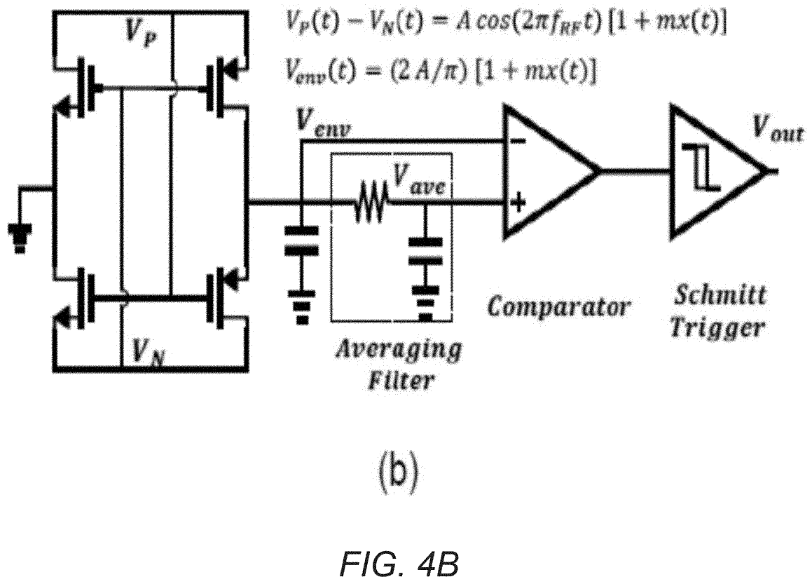

A circuit schematic of an on-chip data transmitter and receiver in accordance with an embodiment of the invention are illustrated in FIG. 4A and FIG. 4B, respectively. To build a data transceiver compatible with a biochemical sensing system, many embodiments may apply the following changes to both transmitter and receiver circuitries.

In many embodiments, the transmitter block may be equipped with a Non-Return-to-Zero (NRZ) to Return to Zero (RZ) converter to enable UWB data communication based on the received data pattern from the PSIO. An NRZ to RZ converter may be necessary for creating a rising edge in the power oscillator driver. In addition, many embodiments may increase the pulse-width of the pulses to 50 nS to relax the bandwidth requirements of the external data receiver. Many embodiments may exploit a Manchester encoding scheme in DL to incorporate a digital clock signal into the power link and enable simultaneous DL communication and clock recovery. In many embodiments, the receiver circuitry may be revised to enable a clock recovery function from the Manchester-encoded received signals. Also, the recovered clock should be amplified properly and distributed across the integrated circuits to ensure reliable operation for all the digital blocks. Although FIGS. 4A and 4B illustrate particular circuit schematics for a UWB TX and an ASK data RX, any of a variety of circuit architectures may be specified as appropriate to the requirements of specific applications in accordance with embodiments of the invention.

### On-Chip Wireless Power Harvester

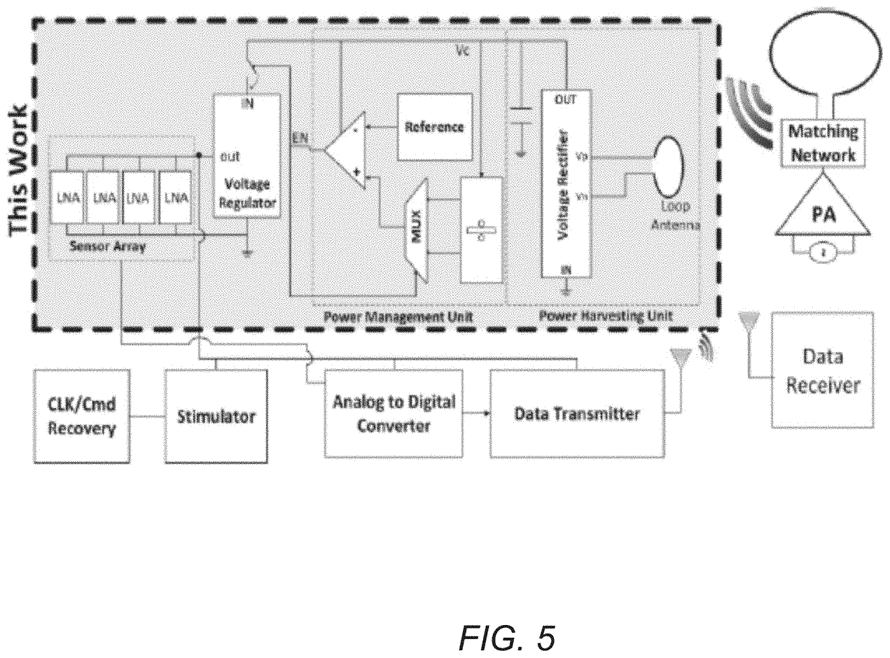

FIG. 5 illustrates a circuit architecture of a power harvesting system that includes a millimeter-sized power receiving coil in accordance with an embodiment of the invention. In many embodiments, energy can be transferred to the system through an inductive radio frequency (RF) link and is converted to a dc voltage by a full-wave voltage rectifier stage. In many embodiments, the system can include a power management unit, which can make the power harvesting system resilient to non-idealities of the wireless link such as antenna misalignment and composition of the intervening medium in the wireless link. A maximum continuous received power can be controlled by a Specific Absorption Rate (SAR), which may limit the thermal absorption of biological tissues to e.g., 1.6 W/kg when exposed to electromagnetic radiation. Based on analysis, a received power by a mm-sized coil may be limited to ˜100 which can be sufficient. Power-demanding blocks of a biochemical sensor may include the instrumentation amplifier, ADC, and wireless transceiver. In many embodiments, the combined power consumption of these blocks may require few milli-Watts of instantaneous power. A power management unit, as shown in FIG. 5 in accordance with an embodiment of the invention, can establish a duty-cycled operation for power-hungry blocks and enables a complex biochemical sensor to operate under stringent power requirements of an RF link. Although FIG. 5 illustrates a particular circuit architecture for a high-performance biochemical sensor, any of a variety of circuit architectures may be specified for the biochemical sensors as appropriate to the requirements of specific applications in accordance with embodiments of the invention.

Although batteries can be a solution for a wireless sensor, the addition of a battery to the system can dramatically increase the cost of the system and reduce the disposability of the electrochemical sensor. Hence, many embodiments of the biosensor platform can utilize wireless power delivery to realize a fully wireless operation. Many embodiments of the biosensor platform can use one or more near-field and far-field power harvesting platforms for building miniaturized biosensors. Many embodiments may utilize a wirelessly powered sensor operating at a distance of about 50 m. The long operating range can help reduce the health risk to the operator. If the efficiency of the wireless power becomes low and the harvested power becomes insufficient, many embodiments may address this issue by thinning the lossy silicon substrate. A cause of a poor wireless power transfer efficiency (other than the operating distance) can be the degraded performance of the power coil caused by a lossy silicon substrate. Accordingly, many embodiments of the biosensor platform can use wafer thinning techniques to reduce the thickness of the fabricated chips to ˜15 μm. In many embodiments, substrate thinning can improve the quality-factor of the power receiving coil. Simulation results reveal that reducing the substrate thickness from 300 μm to 15 μm improves the link efficiency by 9 dB. As a result, the operating range can be extended by a factor of ˜×8. In many embodiments, substrate thinning can make a fabricated chip into a flexible sheet format that can be attached to the skin as a patch and controlled through a wireless, contactless signal. Many embodiments of the biosensor platform may use a small battery to operate the system and still demonstrate a low-power wireless electrochemical sensor that can detect and quantitatively measure IgG/IgM in a wireless fashion.

### Biosensors for Real-Time Glucose Monitoring

Real-time continuous monitoring of glucose can be crucial to alert diabetic patients during actual or pending glycemic excursions, providing timely treatment. However, existing self-monitoring blood glucose systems (SMBG) involve pain and discomfort associated with regular finger-pricking. Continuous Glucose Monitoring (CGM) is an emerging technology that continuously measures interstitial glucose levels, helping maintain a robust glycemic control without augmenting the risk of hypoglycemia. In addition, the continuous monitoring mechanism can effectively detect glucose excursions during atypical times, such as the first few hours following a meal and overnight, which is a vulnerable time for hypoglycemic seizures. Presently, CGM systems generally employ a micro flexible sensor inserted or implanted under the skin, measuring interstitial fluid (ISF) glucose concentration every few minutes and transmitting the data wirelessly to monitor glucose level changes from hours to days.

Current implantable real-time CGM systems are termed “minimally invasive”; they insert a needle coupled with wire type sensor into the skin to measure glucose concentration in ISF without drawing blood. CGM systems still present some limitations, such as lag time, biofouling, fibrous encapsulation of the implanted electrode, battery failure, inflammation, and loss of host vasculature, which can seriously affect the precision and accuracy of the B.G. results. The value displayed on the CGM receiver typically lags capillary blood glucose by an average of 15-20 minutes, where lag caused by sensor membrane is 1-2 minutes, and delay due to CGM device accounts for 3-12 minutes, including filter used for noise reduction. The troubles related to CGM systems include battery and power units in CGM resulting in insulin pump failure, early termination of battery life (within 72 h of use), alarm set off, battery compartment-related problems, and other defects. The association for innovative diabetes treatment of Japan (ASINDTJ) found that battery and power-related trouble cases account for 20-40% of CGM-related failures. Battery and power unit failure in CGM devices are also related to insulin pump malfunction, resulting in hyperglycemic seizures and diabetic ketoacidosis. Accordingly, many embodiments provide for an implantable CGM device that is battery-less and can operate with energy harvesting and that alleviates the worries about the medical device running low on batteries and perhaps missing out on a critical piece of health information being captured and communicated timely. Furthermore, battery-free devices negate the potential risk of lithium poisoning or leakage-related adverse issues.

### Glucose Monitoring Biosensor Architecture

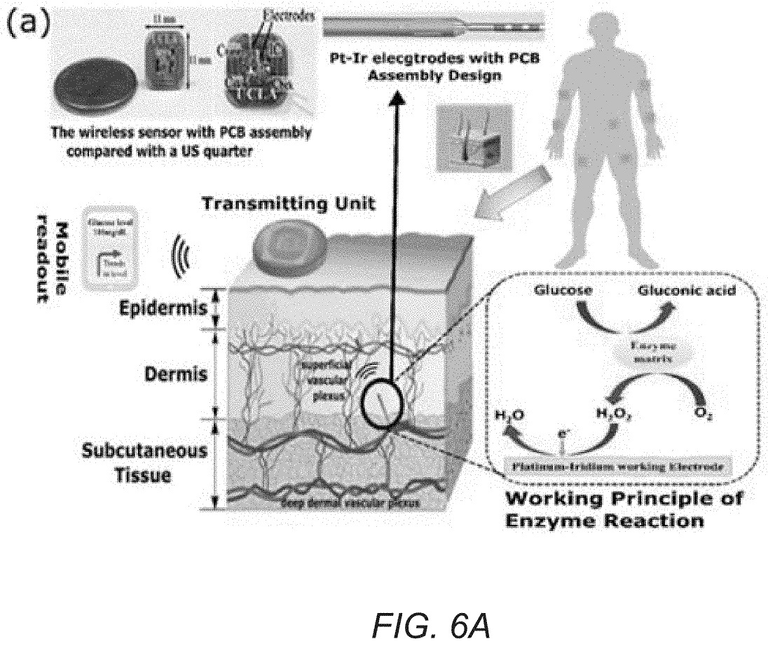

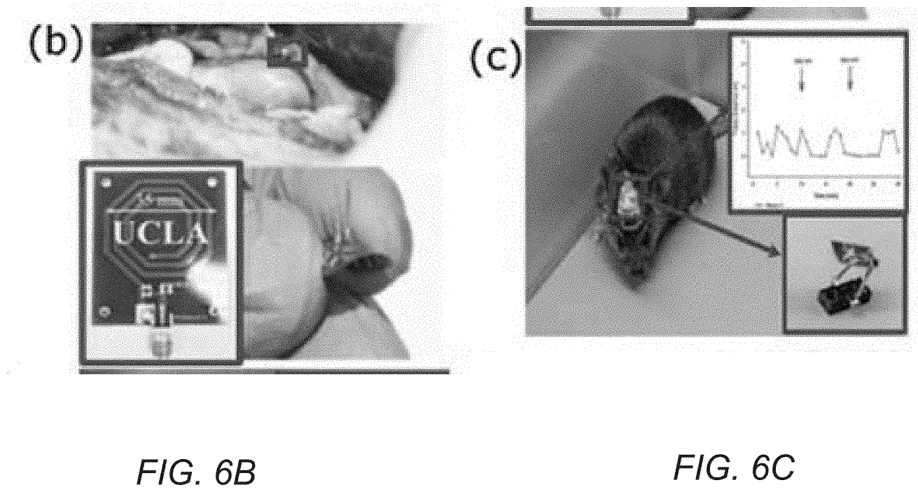

Through glucose-sensing and application, a biosensor platform in accordance with many embodiments can allow patients to monitor blood glucose levels in a fully automated continuous fashion without a trained technician's involvement or fear of battery-related hardware failure. A glucose monitoring biosensor platform architecture in accordance with an embodiment of the invention is illustrated in FIG. 6A.

In particular, FIG. 6A illustrates a fully wireless and batteryless continuous glucose monitoring (CGM) system in accordance with an embodiment of the invention. The CGM system can be adapted and integrated with wirelessly powered technology. FIG. 6B illustrates fabricating custom miniaturized fully functioned batteryless and wirelessly pacemakers with needle-type electrodes in rabbits and open chest porcine animal models. Many embodiments of the biosensor system can provide a miniaturized, lightweight, and flexible design with minimized invasiveness for cardiac implantation. Such a low-power integrated circuit coupled with portable circuit assembly (PCB) can reduce the incident power requirement and extend the wireless operation distance. Many embodiments of the wirelessly powered biosensors can also be applied to CGM systems, including deep brain stimulation (DBS) on freely moving rats via wireless and battery-free continuous neuromodulation system using biocompatible, implantable Pt—Ir (9:1) electrodes. FIG. 6C illustrates a wireless system with PCB and output data in accordance with an embodiment of the invention.

Many embodiments of the glucose monitoring system can inform people about possible nocturnal hypoglycemic seizures (or hyperglycemic events post-dinner) with an abnormal BG level in their body fluids. The sensor platform in accordance with many embodiments can be fully functional at a certain depth (e.g., 4 mm) beneath the skin and can be easily accessed by many people for rapid, inexpensive glucose monitoring. In many embodiments, the biosensor platform can also be coupled with a commercially available portable insulin pump. A fully implantable biosensor device in accordance with an embodiment of the invention is illustrated in FIG. 6B and FIG. 6C. Many embodiments can provide a power harvesting pacemaker chip at 40.68 MHz and 13.54 MHz using an inductive wireless link. The chip performance can be validated up to 8.5 cm and 11 cm distance, respectively, and the Specific Absorption Rate (SAR) generated by the link (0.04 W/Kg) can be 2-3 orders of magnitude lower than certain safety regulation limits (e.g., 10 W/Kg).

Many embodiments provide a wireless minimally-invasive electrochemical sensor with high adaptability, low power, and low noise for robust and quantitative sensing of glucose in interstitial body fluid with high precision.

The sensor platform design in accordance with many embodiments can be a fully battery-less system. In many embodiments, the sensor can be designed as a battery-less system with on-site sensing capability to be implanted with minimally invasive technique on the subcutaneous space and transmit data back to a receiver wirelessly, mounted on the top of the skin. Due to the wireless data transfer capability of the sensor platform, the receiver can be integrated with, for example, rings, watches, phones, among other devices, and may avoid the need for adhesive, thus preventing any possibilities of skin irritation. In many embodiments, a receiver can transfer data to a mobile device (e.g., smartphone, smartwatch, among others) within a certain distance (e.g., 30 feet).

### Glucose Sensor System Design

According to the working principle, glucose sensors can be of different types, e.g., optical, electrochemical, piezoelectric, or magnetic. Many embodiments of the glucose sensor can be an (a) electrochemical glucose sensor with a three-electrode setup, (b) a readout interface that can respond electrically to redox reaction with glucose, and (c) an interface that can process, store, and transmit signals wirelessly from subcutaneous space (e.g., as done in rodent and murine animal models).

### Electrochemical Glucose Sensor Electrode Interface Designs

Many embodiments of the biosensor system can include an electrode interface for an electrochemical amperometric glucose sensor for ultrasensitive human glucose level detection using a Pt—Ir (9:1) wire-based glucose oxidase (GOₓ)-enzyme based complex (Pt-GOₓ) as a label. In many embodiments, the sensing can consume as low an amount of sample as possible and be scalable, rapid, and inexpensive to detect multiple glucose levels at multiple sites in the body. Prior work include a Pt—Ir wire-based glucose sensor that was fabricated for the baseline blood glucose detection of ˜4.45 mmol/L and ˜6.52 mmol/L in subcutaneous space of rats. The glucose sensor enzyme block can be synthesized by incorporating a GOₓ enzyme and phenylenediamine, bovine serum albumin (BSA) using glutaraldehyde (GA) matrix body. Such a complex matrix can then be coated on the Pt wire electrode. Glucose sensor coated with polyurethane (PU) as outer semi-permeable membrane showed higher in-vivo sensitivity >40 nA/mmol and a low detection limit of (4.35 mmol/L) in the rat blood, with less tissue integration, which can be vital for clinical purposes.

Accordingly, many embodiments of the biosensor system can fabricate an electrochemical glucose sensor by immobilizing the BSA/GA/GOₓ enzyme complex on a platinum-iridium wire (e.g., diameter:125 μm, Pt:Ir=9:1) in a 4-electrode assembly. Many embodiments of the biosensor system can use a 3-electrode arrangement for electrochemical testing. Certain embodiments of the biosensor system can include one (or more) extra electrode that can allow for the integration of one extra glucose sensor or lactate sensor for simultaneous measurements. The reference electrode can be obtained by coating one electrode in Silver-Silver Chloride (Ag—AgCl) inks. The sensing electrode can be polished, cleaned, and then electro-coated with a poly (m-phenylenediamine) (PmPD) membrane.

In many embodiments, the enzyme-matrix mixture, which in many embodiments is the BSA/GA/GOₓ mixture itself, can be immobilized on the PmPD coated electrode. The GA is an amine cross-linker that links GOx and BSA, stabilizing the GOx enzyme (Pt—Ir/PmPD/BSA/GA/GOₓ). The GOx oxidizes the glucose to gluconic acid and reacts with oxygen (02) to form hydrogen peroxide (H₂O₂) as illustrated in FIG. 6A. The H2O2 can then be electrochemically oxidized at Pt—Ir surface by a standard bias potential (˜+0.7V) to provide a measurable current proportional to glucose concentration. The enzyme-coated electrode can then again be coated with an epoxy-bound PU membrane by dipping it in an epoxy-PU mixture and then sterilized and dried.

After enzyme immobilization, each target glucose concentration can be quantified by the change in current upon varying glucose concentration using amperometry in a benchtop setting. Many embodiments of the biosensor platform can provide a detection limit of 20-400 mg/dL analogous to human ISF and serum samples and optimize sensors for long-term use in animals (e.g., >6 months).

As described, many embodiments of the biosensor platform can integrate wireless and battery-free readout electronics to enable sensitive and low-cost CGM devices. Many embodiments provide a wirelessly powered glucose sensor based on the above protocols to determine both ISF and serum glucose levels against average and hyperglycemic levels similar to the human body by an electrooxidation of glucose at enzyme captured reaction sites.

### Electrode Fabrication Processes

Electrode fabrication processes in accordance with several embodiments of the invention are described. In certain embodiments, the following steps can be performed in the fabrication processes.

Step 1—Electrode Preparation Before Enzyme Immobilization: Pt—Ir capture platforms can be prepared by stripping off the Teflon cover using a steel blade from both recording and non-recording sides. Then both sides can be slightly polished and cleaned up by sonication, followed by electrochemical cycling in an acidic solution. After cleaning, electrodes can be dried in the oven and electrochemically cycled in mPD solution for PmPD coating. The PmPD coating can enhance the H₂O₂ sensitivity and provide anti-interference abilities to the interferents.

Step 2—Immobilizing Functional BSA/GA/GOₓ Enzyme Complex as a Matrix: The GOₓ enzyme can be mixed in a conditioned BSA/GA mixture and centrifuged to form the desired enzyme-matrix aliquot. The enzyme aliquot contains 1 wt % GOₓ, 4% (v/v) BSA, and 0.6% (v/v) GA. A microsyringe can be used to coat those electrodes with the enzyme aliquot (made in Step 1) under a stereomicroscope using cotton fibers. The coating process can be repeated 2 to 6 times after checking the enzyme layer consistency under the microscope. The enzyme-coated Pt—Ir electrode can be dried for 30-60 mins.

Step 3—Addition of Outer PU Membrane: Before coating the outer membrane, the enzyme-coated electrodes can be dip-coated with Nafion and subjected to thermal drying at 120° C. for 1 h. Nafion can mitigate interferents with anionic nature, e.g., acetaminophen, uric acid, ascorbic acid, which may contribute to the glucose signal, thus adversely affecting the sensor's accuracy and precision. After drying the Nafion-coated electrodes, the outer membrane can be coated in a solution of PU (2% (w/v)) with epoxy adhesive combined with a surfactant agent in tetrahydrofuran (THF). Then all sensors can be kept for 24 h at room temperature and sealed with a brush on electrical tape. After taping, sensors can be stored at 4° C. The PU coating can provide compactness to the overall enzyme layer and less fibrous encapsulation on the sensor surface, increasing the possibility of in-vivo sensing lifetime of implantable glucose biosensors.

Step 4—Storage of Glucose Sensor Before Being Tested: Before using glucose, sensors can be sterilized by placing it in a 5 mM glucose conditioning solution for 2 days. After conditioning, the glucose-sensing electrodes can be biased at ˜+0.7V vs. SCE for minimum background current.

Step 5—Calibration and Selectivity of Glucose Sensor: The sensor can be first calibrated in glucose-only solution for a range of concentrations, e.g., 20-400 mg/dL, allowed within the capillary blood glucose range at 37° C. As described previously, the sensor interference can be assessed with and without galactose. In addition, interference can be assessed in various sugar alcohol, artificial sweetener, and other non-glucose sugar solutions. The sensor's selectivity can also be assessed by comparing the response to glucose in the presence of ascorbic acid, acetaminophen, and uric acid along with creatinine, triglycerides, bilirubin.

Alternatives: The detection limits and sensitivity and specificity profiles of the glucose sensor in accordance with many embodiments can depend on the enzyme layer stability, glucose limiting capability, and interference rejection. For example, the PU outer membrane can provide compactness to the whole enzyme layer so that enzyme is not leached out of the sensor. However, excess diffusion of glucose from ISF may prematurely saturate the enzyme layer and affect glucose sensing. In such cases, a glucose limiting membrane consisting of polyethyleneimine (PEI) cross-linked with a tri-epoxide linker, trimethylolpropane triglycidylether (PTGE), can limit glucose diffusion, thus improving sensing time. In addition, if Nafion coating is ineffective in restricting neutrally charged acetaminophen, the composite Nafion and cellulose acetate membranes can be used as an alternative interference rejecting layer as described above in step 3.

In many embodiments, analytical calibration, specificity, and sensitivity of a glucose sensor can be assessed using potentiostat in the early electrochemical characterization. The sensing capabilities of the wireless and battery-free system and the ability to determine the lower limit of glucose corresponding to the previous tests (compared to the anticipated 60 mg/dL range of published values for glucose levels into human serum for severe hypoglycemia) can be determined as the sensor get validated having an estimated response to rapid change in glucose concentration (e.g., <2 mg/dL/min).

Wireless and Battery-Free Readout Electronics with Sensitive and Continuous Glucose Monitoring

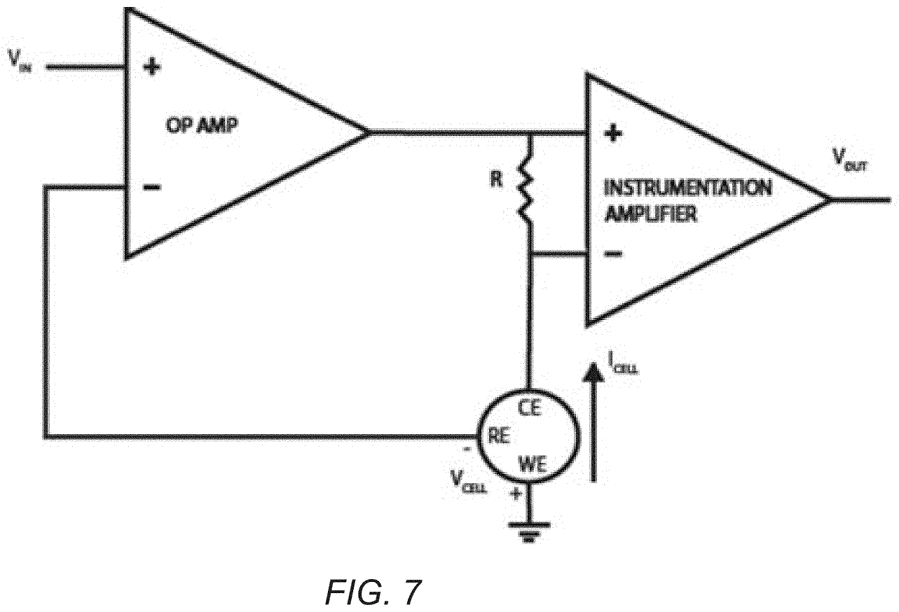

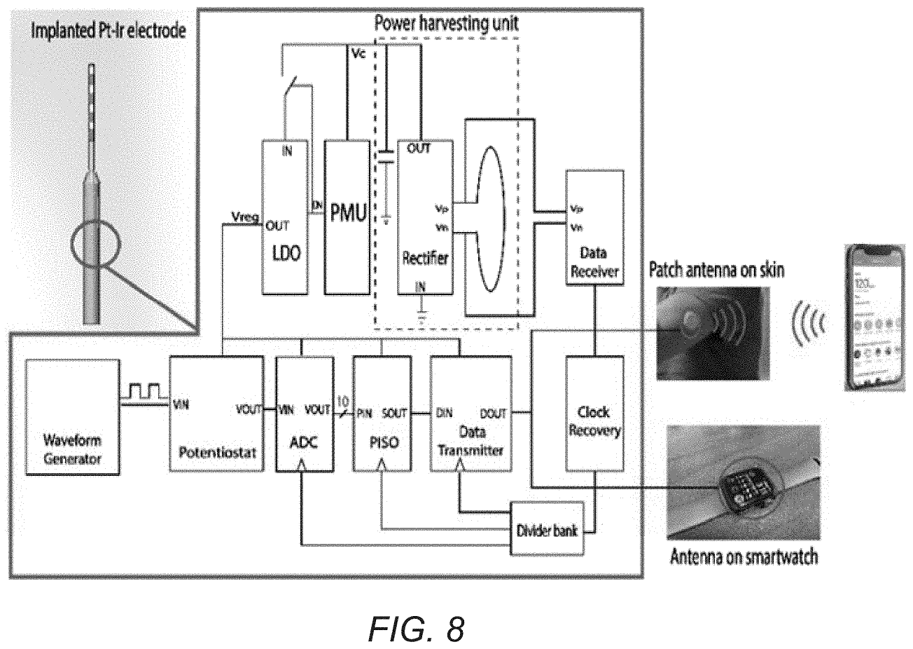

Many embodiments of the biosensor platform provide an integrated signal conditioning and data communication microchip that includes: (1) a potentiostat amplifier (as illustrated in FIG. 7 in accordance with an embodiment of the invention); (2) A high-resolution Analog to Digital Converter (ADC) (3) A low-power Parallel Input Serial Output (PISO) memory; (4) An ultra-low-power data receiver and a clock recovery sub-system; and (5) An ultra-low power data transmitter. A microchip architecture of an implantable CGM in accordance with an embodiment of the invention is illustrated in FIG. 8.

In many embodiments, the microchip can be integrated and attached to an electrode interface as described above. In many embodiments, the biochemical electrodes that can be utilized for glucose level detection can have low driving current and high output impedance. Hence, the output signal of the electrode may not be directly used for driving data communication circuitry that loads the preceding stages. Also, the ambient noise sources of the power link may overwhelm the output signal of the electrode. Accordingly, many embodiments of the biosensor platform can include a potentiostat amplifier. A circuit schematic of potentiostat amplifier in accordance with an embodiment is illustrated in FIG. 7. The functionalities of the potentiostat amplifier can include: a) maintaining a fixed voltage between the working and reference electrodes equal to the potential of the electrochemical cell, which supplies the current necessary for the electrochemical reaction and b) measuring the current flowing through the electrochemical cell, converting it to voltage and finally boosting the voltage so that it is at an acceptable level for driving an ADC. Part (a) can accomplished using a low-power operational amplifier-based voltage follower configuration, while part (b) may use a low-power instrumentation amplifier.

In many embodiments, the instrumentation amplifier featuring a fully differential design can achieve a Common Mode Rejection Ratio (CMRR) of e.g., 60 dB, which can result in the reduction and possible elimination of the common mode noise sources. Furthermore, to amplify the low-frequency output voltage of the electrode, many embodiments of the biosensor system include an on-chip instrumentation amplifier. The biosensor platform can be used to implement an integrated pH sensor achieving a variable gain of up to e.g., ×50 with a power consumption of less than e.g., 10 μW.

In many embodiments, the analog output of the on-chip instrumentation amplifier can be converted to digital bits to preserve data accuracy during transmission, enabling low-power wireless communication. Many embodiments of the biosensor platform include a low-power on-chip ADC in the design with a e.g., 10-bit resolution to enhance the overall detection accuracy of the biochemical sensor. By adopting a set-and-down architecture, the overall size of an ADC can be reduced in half. In many embodiments, an ADC can be implemented with a unit capacitor size of 17.5 fF achieving a power consumption of 300 nW for a sampling rate of 10 KSps. Simulated results show that the an ADC can achieves a Signal-to-Noise-Distortion-Ratio (SNDR) of 67.8 dB and an Effective Number of Bits (ENoB) of 9.3. In many embodiments, the digitized data can be loaded in a PISO memory and fed to a data transmitter serially. The PSIO can be implemented as a digital shift register. The on-chip wireless radio can be a task that dominates the power consumption and limits the operating range. Active radios have been demonstrated with >10 Mbps data rate and >5 cm operating range recently. However, off-chip components used for power delivery and data communication have limited integration capability of these devices. Accordingly, many embodiments of the biosensor platform provide an integrated radio with a millimeter-sized form factor. In particular, many embodiments of the biosensor platform include an integrated wirelessly powered radio providing a fully integrated wireless sensor. In many embodiments, the wirelessly powered radio can be integrated with on-chip antennas that consume, for example, a total volume of e.g., 2.4×2.2×0.3 mm³, achieving 2.5 Mbps downlink (DL) D.R. with 1.04 pJ/b efficiency, maximum uplink (U.L.) D.R. of 150 Mbps under duty-cycled operation, and a maximum U.L. energy efficiency of 4.65 pJ/b.

In many embodiments, the SAR can control a maximum continuous received power, limiting the thermal absorption of biological tissues to e.g., 1.6 W/kg when exposed to electromagnetic radiation. In many embodiments, the received power by a millimeter-sized coil can be limited to ˜100 μW, which can be sufficient. Power-demanding blocks of a biochemical sensor can include the instrumentation amplifier, ADC, and wireless transceiver. The combined power consumption of these blocks may require few milli-Watts of instantaneous power. In many embodiments, a power management unit can establish a duty-cycled operation for power-hungry blocks and enable a complex biochemical sensor to operate under the stringent power requirements of an RF link.

Electrochemical Sensor Architecture with Power Management Unit

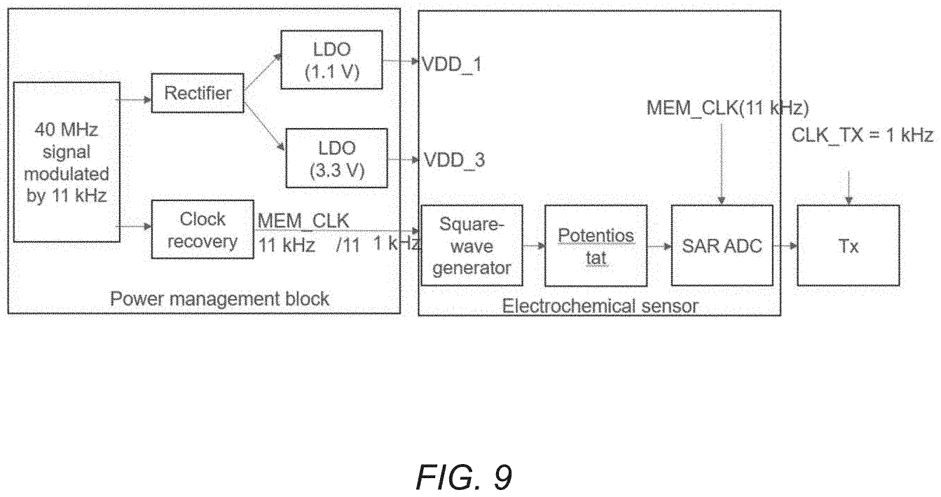

Electrochemical sensor platforms in accordance with many embodiments can use different types of voltammetry with a potentiostat. A circuit architecture of a electrochemical sensor platform with a square-wave generator in accordance with an embodiment of the invention is illustrated in FIG. 9. The sensor includes a power management block and an electrochemical sensor. In many embodiments, the electrochemical sensor can use a square-wave voltammetry to provide improved sensitivity. Voltammetry can describe the non-spontaneous interfacial charge transfer process that is driven by externally applied electrical potential difference in an electrolytic cell. Different types of voltammetry can include linear sweep voltammetry (LSV) and cyclic voltammetry (CV). As illustrated in FIG. 9, the square-wave generator can generate an output waveform that is provided to a potentiostat. Although FIG. 9 illustrates particular circuit architecture of a electrochemical sensor, any of a variety of circuit designs can be utilized as appropriate to the requirements of specific applications in accordance with embodiments of the invention.

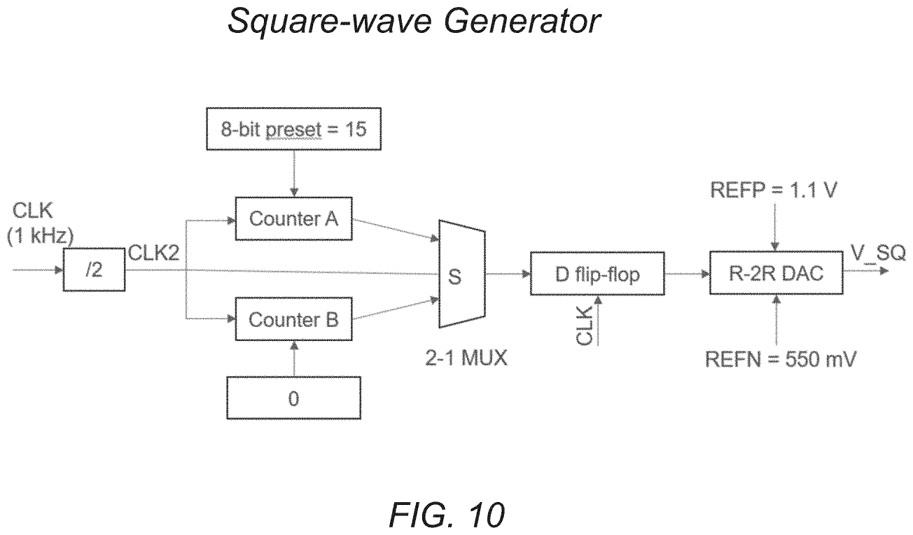

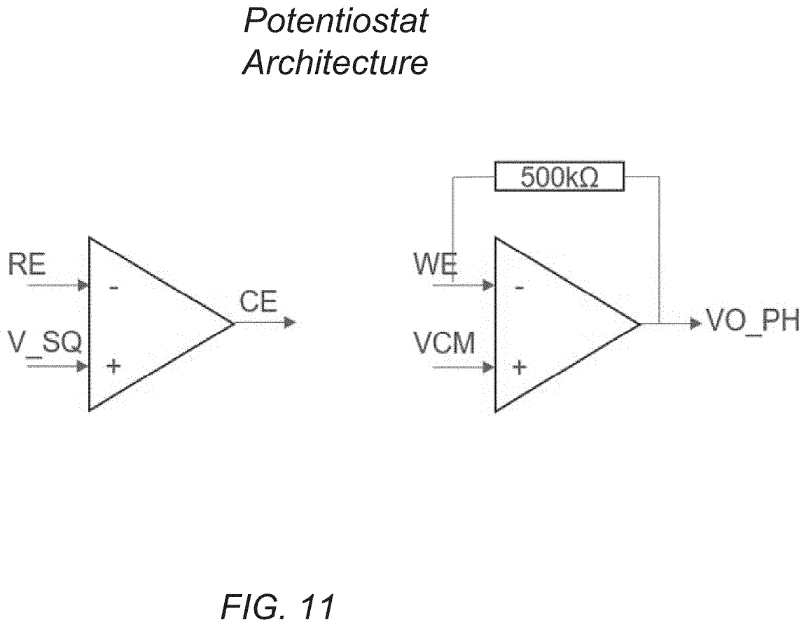

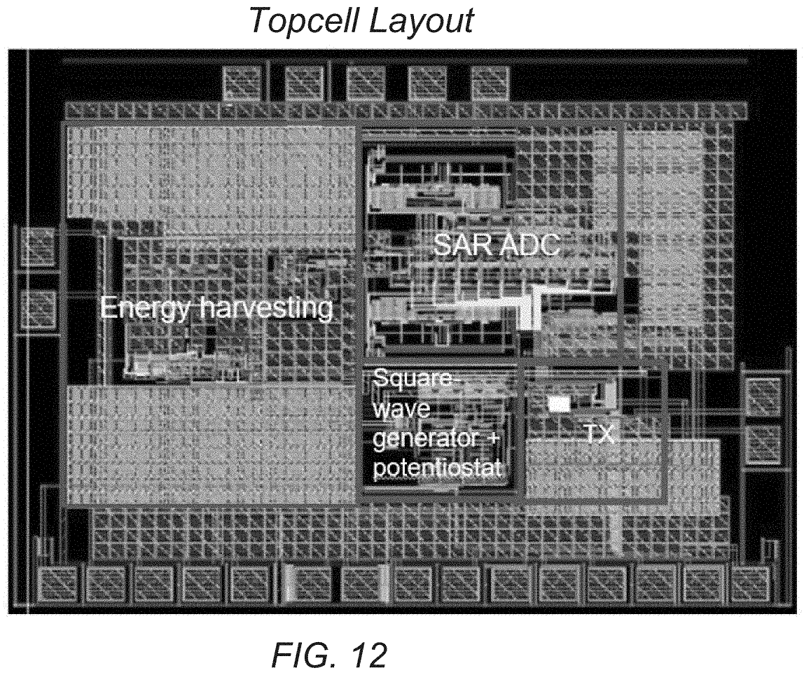

A circuit architecture of a square wave generator in accordance with an embodiment of the invention is illustrate in FIG. 10. Although FIG. 10 illustrates particular circuit architecture of a square-wave generator, any of a variety of circuit designs can be utilized as appropriate to the requirements of specific applications in accordance with embodiments of the invention. A circuit architecture of a potentiostat in accordance with an embodiment of the invention is illustrate in FIG. 11. Although FIG. 11 illustrates particular circuit architecture of a potentiostat, any of a variety of circuit designs can be utilized as appropriate to the requirements of specific applications in accordance with embodiments of the invention. A circuit architecture of a electrochemical sensor in accordance with an embodiment of the invention is illustrate in FIG. 12. As illustrated, the circuit includes an energy harvesting unit that harvest wireless power from an external source, a SAR ADC, a square wave generator, a potentiostat, and a transmitter (TX) that communicates wirelessly with an external controller. Although FIG. 12 illustrates particular circuit architecture of a electrochemical sensor, any of a variety of circuit designs can be utilized as appropriate to the requirements of specific applications in accordance with embodiments of the invention.

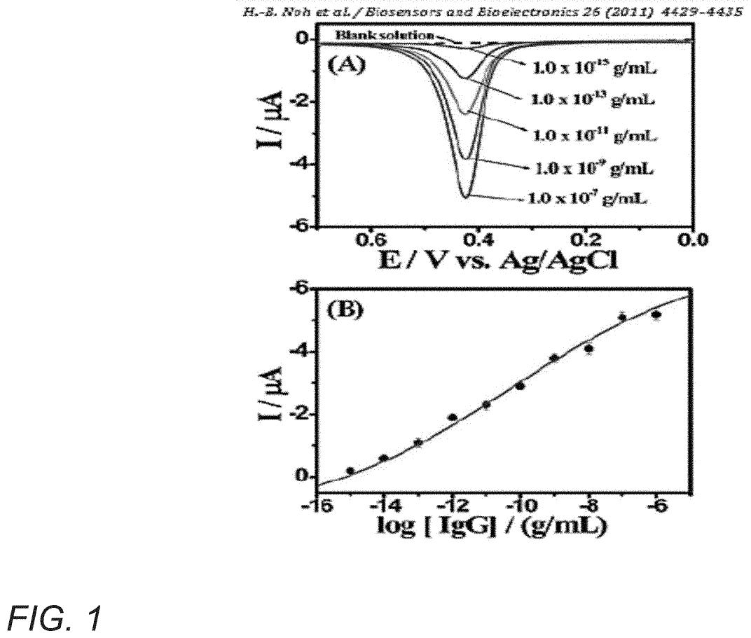

Although specific implementations for wirelessly powered biosensors are discussed above with respect to FIGS. 1-12, any of a variety of implementations utilizing the above discussed techniques can be utilized for wirelessly powered biosensors in accordance with embodiments of the invention. While the above description contains many specific embodiments of the invention, these should not be construed as limitations on the scope of the invention, but rather as an example of one embodiment thereof. It is therefore to be understood that the present invention may be practice otherwise than specifically described, without departing from the scope and spirit of the present invention. Thus, embodiments of the present invention should be considered in all respects as illustrative and not restrictive.

What is claimed is:

1. A wirelessly powered electrochemical sensor system, comprising:
an external controller;
an implantable microchip that includes an electrochemical sensor coupled to a target molecule;
wherein the microchip communicates with the external controller and receives wireless power from the external controller;
wherein the microchip measures at least one of current and voltage from the electrochemical sensor.

2. The wirelessly powered electrochemical sensor system of claim 1, wherein the harvested wireless power is stored in a capacitor.

3. The wirelessly powered electrochemical sensor system of claim 2, wherein the stored energy in the capacitor is used to generate a regulated voltage to activate the electrochemical sensor.

4. The wirelessly powered electrochemical sensor system of claim 1, wherein the harvested wireless power is received by at least one of a loop antenna, dipole antenna, resonant loop antenna, inductive coupling, and resonant inductive coupling.

5. The wirelessly powered electrochemical sensor system of claim 1, wherein a frequency of the wireless power is between 1 MHz and 1 GHz.

6. The wirelessly powered electrochemical sensor system of claim 1, wherein the physical distance between the implantable microchip and external controller is smaller than a wavelength of the electromagnetic signal used for wirelessly powering the microchip.

7. The wirelessly powered electrochemical sensor system of claim 1, wherein a plurality of commands are sent by the external controller and are encoded on the RF carriers by means of amplitude shift keying (ASK), frequency shift keying (FSK), phase shift keying (PSK), on-off keying (OOK), or other complex coding schemes.

8. The wirelessly powered electrochemical sensor system of claim 1, wherein an electrochemical signature measured by the implantable microchip is transmitted wirelessly to the external controller.

9. The wirelessly powered electrochemical sensor system of claim 8, wherein the electrochemical signature is digitized by an ADC before being transmitted to the external controller.

10. The wirelessly powered electrochemical sensor system of claim 1, wherein the electrochemical sensor is a glucose sensor that monitors glucose levels.

11. The wirelessly powered electrochemical sensor system of claim 10, wherein the glucose sensor comprises:
an electrochemical glucose sensor with an implantable electrode with a three-electrode setup;
a readout interface that responds electrically to redox reaction with glucose;
an interface that processes, stores, and transmits signals wirelessly from subcutaneous tissue of the patient.

12. The wirelessly powered electrochemical sensor system of claim 11, wherein the electrochemical glucose sensor is a 4-electrode setup, wherein 3 electrodes in the setup test electrochemicals and a 4^{th} electrode in the setup is at least one of a glucose sensor and lactate sensor that generates simultaneous measurements.

13. The wirelessly powered electrochemical sensor system of claim 11, further comprising:
an on-chip analog to digital converter (ADC);
a potentiostat amplifier that maintains a fixed voltage between working and reference electrodes in the three-electrode setup equal to the potential of the electrochemical cell; and
wherein the potentiostat amplifier measures current flowing through the electrochemical cell, converts the current to voltage, and boosts the voltage to drive the on-chip ADC.

14. The wirelessly powered electrochemical sensor system of claim 13, further comprising an on-chip instrumentation amplifier that amplifies a low-frequency output voltage of implantable electrode, wherein an analog output of the on-chip instrumentation amplifier is converted to digital bits using the on-chip ADC.

15. The wirelessly powered electrochemical sensor system of claim 1, wherein the external controller is positioned on or near a patient's skin and the implantable microchip is implanted beneath the skin.

16. The wirelessly powered electrochemical sensor system of claim 1, wherein the external controller is an application executing on a mobile device.

17. The wirelessly powered electrochemical sensor system of claim 1, further comprising applying a voltage between a reference electrode and a working electrode while reading a current from a counter electrode.

18. The wirelessly powered electrochemical sensor system of claim 1, further comprising sweeping the voltage and reading of the current to plot a current versus voltage curve, wherein the curve is used to identify different biomolecules.

19. The wirelessly powered electrochemical sensor system of claim 1, wherein the microchip further comprises:
an on-chip RF-power receiving antenna that receives electromagnetic power from the external controller;
an on-chip transmitting antenna that transmits data to the external controller;
a power management unit (PMU) comprising a capacitor to rectify and store the electromagnetic power, wherein the PMU monitors harvested energy and operates the electrochemical sensor.

20. The wirelessly powered electrochemical sensor system of claim 1, wherein the electrochemical sensor detects COVID-19 reactive antibodies of the IgM and IgG isotypes.
